(19) United States
(12) Patent Application Publication
(10) Pub. No.: US 2026/0335133 A1
(43) Pub. Date: Oct. 29, 2026

(54) SYSTEMS AND METHODS FOR DETECTING ZONULAR APPARATUS INSTABILITY

(71) Applicant: Regents of The University of Michigan, Ann Arbor, MI (US)

(72) Inventors: Nambi Nallasamy, Ann Arbor, MI (US); Binh Duong Giap, Ann Arbor, MI (US)

(21) Appl. No.: 19/658,125

(22) Filed: Apr. 24, 2026

Related U.S. Application Data
(60) Provisional application No. 63/794,907, filed on Apr. 25, 2025.

(51) Int. Cl. A61F 9/007; A61B 34/10; A61B 90/00

(52) U.S. Cl. CPC A61F 9/00754; A61B 34/10; A61B 90/361; A61B 2034/108

(57) ABSTRACT

System and methods for detecting an instability of a zonular apparatus of an eye of a subject using machine learning are provided. An example system may obtain surgical image data comprising a plurality of images of the eye of the subject captured during an ophthalmic surgical procedure. The system may provide the surgical image data to a machine learning model trained to determine the instability of the zonular apparatus based upon analyzing the surgical image data including identifying at least one characteristic of the eye indicating the instability of a zonular apparatus, and performing a zonular apparatus weakness analysis. Based upon the zonular apparatus weakness analysis, the system may generate output data including information associated with surgical support of the zonular apparatus, and transmit the output data to a computing device.

### CROSS-REFERENCE TO RELATED APPLICATIONS

This application claims the benefit of U.S. Application No. 63/794,907, filed Apr. 25, 2025, and entitled “Systems and Methods for Detecting Zonular Apparatus Instability”, which is incorporated herein by reference in its entirety.

### GOVERNMENT LICENSE RIGHTS

This invention was made with government support under TW012027, and EY022299 awarded by the National Institutes of Health. The government has certain rights in the invention.

### FIELD OF THE DISCLOSURE

The present disclosure generally relates to systems and methods for analyzing surgical images, and more particularly, to systems and methods for using machine learning to detect zonular apparatus instability during an ophthalmic surgical procedure.

### BACKGROUND

The zonular apparatus of the natural crystalline lens in the human eye is responsible for suspending the lens via zonular fibers in a position posterior to the iris. Loss of the zonular fibers may occur due to trauma, prior surgical procedures (e.g., pars plana vitrectomy, intravitreal injection), and/or a medical condition (e.g., pseudoexfoliation syndrome, Marfan syndrome, homocystinuria, Ehlers-Danlos syndrome, high myopia), causing zonular apparatus weakness that allows the lens to shift or otherwise causes instability of the lens. A surgical subject affected by zonular apparatus weakness or instability may be at an increased risk of severe complications during cataract surgery, such as losing the entire capsule or lens fragments behind the posterior segment of the eye.

Conventional methods of addressing zonular apparatus instability include the surgeon visually detecting one or more signs of zonular apparatus weakness of the subject early on in the cataract surgery, so the surgeon may alter their surgical strategy to address the zonular apparatus instability, for example by introducing capsular support devices (e.g., capsular hooks, capsular tension rings, and capsular tension segments) to improve capsular stability before capsular rupture or other complications such as iris trauma, intraocular hemorrhage, vitreous loss, and/or aphakia occur. Thus, the decision-making process to address zonular apparatus weakness generally depends on the subjective assessment and visual acuity of the surgeon, which can vary across different surgical scenarios and conditions of the surgical subject and put the surgical subject at risk for adverse consequences. Unfortunately, when zonular apparatus weakness is not detected early enough, the experience level of the surgeon does not correlate and/or compensate for intraoperative complication rates caused by zonular apparatus weakness which can lead to increased surgical times and heightened risk of intraoperative complications. Accordingly, early interoperative detection and intervention techniques applied in the initial stages of an ophthalmic surgical procedure can be of even greater value than the experience level of the surgeon. Thus, the conventional techniques to address zonular apparatus instability lack objective, real-time, analytics-based techniques for dynamically detecting zonular apparatus weakness which can enable the early stabilization of the lens capsule during surgical operations to reduce risk to the surgical subject.

Given the aforementioned challenges, there are opportunities for systems and methods that provide improved detection of zonular apparatus instability during ophthalmic surgery procedures to reduce the risk of complications to the surgical subject, improve patient outcomes, and otherwise avoid the shortcomings and inconsistencies of the present techniques.

### BRIEF SUMMARY

In one aspect, a computer-implemented method for detecting an instability of a zonular apparatus of an eye of a subject using machine learning may include; (i) obtaining, by one or more processors, surgical image data comprising a plurality of images of the eye of the subject captured during an ophthalmic surgical procedure; (ii) providing, by the one or more processors, the surgical image data to a machine learning model trained to determine the instability of the zonular apparatus based upon analyzing the surgical image data including: (a) identifying at least one characteristic of the eye indicating the instability of a zonular apparatus; and (b) based upon identifying the at least one characteristic of the eye indicating the instability of the zonular apparatus, performing a zonular apparatus weakness analysis; (iii) based upon the zonular apparatus weakness analysis, generating, by the one or more processors, output data including information associated with surgical support of the zonular apparatus; and (iv) transmitting, by the one or more processors, the output data to a computing device. The computer-implemented method may include additional, less, or alternate functionality or actions, including those discussed elsewhere herein.

For example, the computer-implemented method may include performing, by the one or more processors, preprocessing on the surgical image data, including extracting a region of interest (ROI) corresponding to an anterior segment of the eye and performing at least one preprocessing operation selected from resizing, normalizing pixel intensity values, and enhancing image contrast or quality.

In another aspect, a system for detecting an instability of a zonular apparatus of an eye of a subject using machine learning may include: one or more processors; and one or more non-transitory memories storing processor-executable instructions that, when executed by the one or more processors, cause the system to: (i) obtain surgical image data comprising a plurality of images of the eye of the subject captured during an ophthalmic surgical procedure; (ii) provide the surgical image data to a machine learning model trained to determine the instability of the zonular apparatus based upon analyzing the surgical image data including: (a) identifying at least one characteristic of the eye indicating the instability of a zonular apparatus; and (b) based upon identifying the at least one characteristic of the eye indicating the instability of the zonular apparatus, performing a zonular apparatus weakness analysis; (iii) based upon the zonular apparatus weakness analysis, generate output data including information associated with surgical support of the zonular apparatus; and (iv) transmit the output data to a computing device. The system may also perform, by the one or more processors, preprocessing on the surgical image data, including extracting a region of interest corresponding to an anterior segment of the eye and at least one of resizing the surgical image data or the region of interest, normalizing pixel intensity values, or enhancing image contrast or quality.

In another aspect, a non-transitory computer readable storage medium storing executable instructions that, when executed by a processor, cause the one or more processors to at least: (i) obtain surgical image data comprising a plurality of images of an eye of a subject captured during an ophthalmic surgical procedure; (ii) provide the surgical image data to a machine learning model trained to determine instability of a zonular apparatus of the eye based upon analyzing the surgical image data including: (a) identifying at least one characteristic of the eye indicating the instability of a zonular apparatus; and (b) based upon identifying the at least one characteristic of the eye indicating the instability of the zonular apparatus, performing a zonular apparatus weakness analysis; (iii) based upon the zonular apparatus weakness analysis, generate output data including information associated with surgical support of the zonular apparatus; and (iv) transmit the output data to a computing device.

### BRIEF DESCRIPTION OF THE DRAWINGS

The figures described below depict various aspects of the system and methods disclosed therein. It should be understood that each figure depicts one embodiment of a particular aspect of the disclosed system and methods, and that each of the figures is intended to accord with a possible embodiment thereof. Further, wherever possible, the following description refers to the reference numerals included in the following figures, in which features depicted in multiple figures are designated with consistent reference numerals.

There are shown in the drawings arrangements which are presently discussed, it being understood, however, that the present aspects are not limited to the precise arrangements and instrumentalities shown, wherein:

FIG. 1 depicts a block diagram of an example computing environment in which methods and system for detecting zonular apparatus instability are implemented, according to embodiments;

FIG. 2 depicts a combined block and logic diagram for training a machine learning model, according to embodiments;

FIG. 3A depicts an example system for detecting zonular apparatus instability using machine learning, according to embodiments;

FIG. 3B depicts an example first image of an ophthalmic surgical video indicating zonular apparatus instability, according to embodiments;

FIG. 3C depicts an example second image of an ophthalmic surgical video indicating zonular apparatus instability, according to embodiments;

FIG. 3D depicts an example composite image providing surgical guidance, according to embodiments; and

FIG. 4 an example block flow diagram depicting a computer-implemented method for detecting an instability of a zonular apparatus of an eye of a subject using machine learning, according to embodiments.

Advantages will become more apparent to those skilled in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### DETAILED DESCRIPTION

### Overview

The disclosed systems and methods provide techniques for detecting an instability of a zonular apparatus of an eye of a subject using machine learning. The disclosed techniques employ a trained machine learning model to analyze surgical image data comprising images capturing an ophthalmic surgical procedure (e.g., cataract surgery) of a subject to determine the instability of the zonular apparatus. The model may identify at least one characteristic of the eye indicating the instability of a zonular apparatus, and based upon identifying the at least one characteristic, performing a zonular apparatus weakness analysis. Based upon the zonular apparatus weakness analysis, the disclosed systems and methods may generate output data including information associated with surgical support of the zonular apparatus, and transmit the output data to a computing device. In some embodiments, the surgical image data comprises a sequence of temporally ordered frames (e.g., video data). The machine learning model is configured to analyze temporal relationships across frames, including motion, persistence, and deformation patterns of anatomical structures such as the lens, capsular bag, or iris. Temporal analysis enables differentiation between transient surgical artifacts and persistent indicators of zonular instability.

This disclosed systems and methods address shortcomings of convention techniques for detecting zonular apparatus weakness in the field of ophthalmology by providing objective, real-time, analytics-based techniques for dynamically detecting zonular apparatus weakness to enable the early stabilization of the lens capsule during surgical operations to reduce risk to the surgical subject. Unlike conventional techniques which rely on visual acuity and subjective interpretation to identify signs of zonular apparatus instability, a key feature of the present techniques includes the use of machine learning to analyze images of the surgical procedure to detect zonular apparatus instability. Implementing a trained machine learning model offers a high degree of accuracy in detecting zonular apparatus instability throughout the various conditions (e.g., anatomical condition of the patient anatomy, environmental conditions of the surgical environment, etc.) and surgical phases of the ophthalmic surgical procedure, especially initial surgical phases when decisions regarding surgical support of the zonular apparatus and/or lens may be most crucial to avoiding risk to the surgical subject. Accordingly, the ability to accurately identify zonular apparatus weakness during the surgical procedure empowers surgeons to adapt their operating strategies in real-time, potentially reducing surgical complications and associated time to address such complications, and improving the efficiency of surgical procedures.

The disclosed techniques employ a trained machine learning model to analyze surgical images indicative of lens responses to various surgical maneuvers, such as medication and viscoelastic instillation, capsulotomy, phacoemulsification, and irrigation. Through the analysis, the system can detect subtle signs of zonular weakness and even provide surgical guidance, allowing for early intervention to address zonular apparatus instability. The proactive detection of zonular apparatus instability provided by the machine learning model can be critical to enabling the surgical team to adjust their surgical approach before complications arise in the surgical subject, significantly reducing the risk associated with the ophthalmic surgical procedure. By integrating decision support surrounding the utilization of various capsular support devices based on substantially real-time analysis of surgical images, the system not only identifies problems but provides actionable solutions, thereby providing improved mechanisms for supporting intraoperative capsular stability, improving patient surgical outcomes, and overall advancing the field of ophthalmic surgery.

The machine learning-based intervention introduces improved networking and processing capabilities, as the machine learning model is trained to efficiently process and analyze complex surgical image data substantially in real-time while a surgical procedure is on-going. This ability to rapidly interpret data and extract relevant findings regarding zonular weakness surpasses conventional detection methods based upon subjective examination techniques in both speed and accuracy to provide surgeons with critical surgical information in a timely manner. The disclosed systems and methods analyze a multitude of images captured during surgery using machine learning techniques to detect subtle yet critical signs of zonular apparatus weakness, offering a significant leap forward in patient care and surgical outcomes to enhance, rather than burden, existing technological infrastructure of clinical settings. The disclosed systems are configured to provide a combination of advanced image processing, machine learning, and real-time surgical data analysis to identify signs of zonular apparatus instability that may otherwise go unnoticed, allowing for early surgical intervention to reduce the likelihood of associated surgical complications and enhance the overall safety and efficacy of cataract surgery. The disclosed systems and methods not only facilitate the rapid retrieval and analysis of surgical image data, but also provide the ongoing refinement of machine learning models through retraining as additional training data becomes available. Moreover, the ability to provide (near) real-time surgical image analysis reduces the duration and/or number of surgical procedures and associated surgical computing resources (e.g., processing cycles, memory, network bandwidth, power) to otherwise address complications caused by zonular apparatus instability, leading to improved computing performance and better surgical outcomes and patient satisfaction.

In sum, by integrating machine learning and comprehensive image data analysis into the field of ophthalmic surgery, the present techniques represent a substantial advancement in surgical precision and patient safety. This disclosed systems and methods not only enhance the capability of surgeons to identify and address zonular weakness proactively, but also introduce significant computational improvements that streamline surgical processes, making advanced ophthalmic care more accessible and effective. Through these innovations, the present techniques set a new standard for the integration of technology and medicine that present a holistic solution to the aforementioned challenges of convention techniques in effectively detecting zonular apparatus weakness. Through its innovative approach, the disclosed techniques allow for technology-assisted surgical procedures which provide advancements in medical technology applications to prioritize patient safety and surgical success.

As used herein, the terms video data and/or image data may refer to data comprising one or more images (e.g., images of a surgical procedure). Accordingly, at times the terms “video data,” “image data,” and the like may be used interchangeably with the terms “image,” “images,” “video,” and the like. Additionally, the terms “weakness” and “instability” may be used interchangeable herein with respect to the zonular apparatus.

### Example Computing Environment

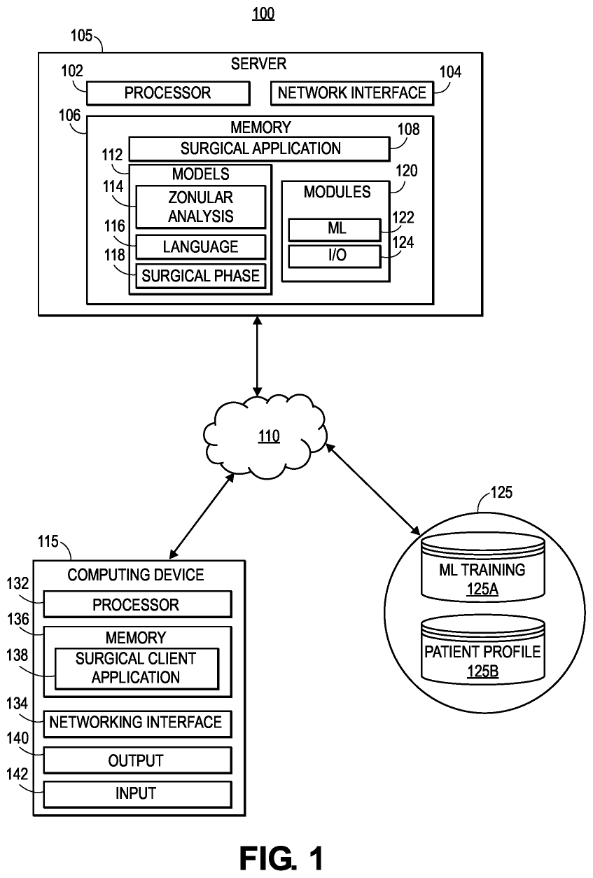

FIG. 1 depicts an example computing environment 100 in which methods and system for detecting zonular apparatus instability using machine learning (ML) may be implemented, according to embodiments. Although FIG. 1 depicts certain entities, components, equipment, and/or devices, it should be appreciated that additional or alternate entities, components, equipment, and/or devices are envisioned.

The computing environment 100 may include at least one server 105 which may perform the at least some of the functionalities and techniques disclosed, such as obtaining surgical image data, executing machine learning models, detecting zonular apparatus instability, generating surgical recommendations, etc. The server 105 may be referred to at times more generically as a “computing device” or “device.” The server 105 may be part of a cloud network or may otherwise communicate with other hardware or software components within one or more cloud computing environments to send, retrieve, or otherwise analyze data or information described herein. In some embodiments, the computing environment 100 may comprise an on-premises computing environment, a multi-cloud computing environment, a public cloud computing environment, a private cloud computing environment, and/or a hybrid cloud computing environment. In one example, an entity may host one or more services (e.g., surgical video analysis) via a public cloud computing environment (e.g., Amazon Web Services (AWS), Google Cloud, IBM Cloud, Microsoft Azure, etc.). The public cloud computing environment may be a traditional off-premises cloud (i.e., not physically hosted at a location owned/controlled by the business). Alternatively, or in addition, aspects of the public cloud may be hosted on-premises at a location owned/controlled by the entity. The public cloud may be partitioned using visualization and multi-tenancy techniques and/or may include one or more of software-as-a-service (SaaS), infrastructure-as-a-service (IaaS) and/or platform-as-a-service (PaaS). In one aspect, the server 105 may include a client-server platform technology such as ASP.NET, Java J2EE, Ruby on Rails, Node.js, a web service or online API, responsive for receiving and responding to electronic requests.

The computing environment 100 may include a network 110 comprising any suitable network or networks, including a local area network (LAN), wide area network (WAN), the Internet, a combination thereof, and/or any other suitable network. Generally, the network 110 enables bidirectional communication between the server 105, the computing device 115, and other components and/or devices of the computing environment 100. In some embodiments, the network 110 may comprise a cellular base station, such as cell tower(s), communicating to the one or more components of the computing environment 100 via wired/wireless communications based upon any one or more of various mobile phone standards, including NMT, GSM, CDMA, UMTS, LTE, 5G, 6G, or the like. Additionally, or alternatively, the network 110 may comprise one or more routers, wireless switches, or other such wireless connection points communicating to the components of the computing environment 100 via wireless communications based upon any one or more of various wireless standards, including by non-limiting example, IEEE 802.11 a/ac/ax/b/c/g/n (Wi-Fi), Bluetooth, and/or the like.

The server 105 may include at least one processor 102. The processor 102 may include one or more suitable processors (e.g., central processing units (CPUs) and/or graphics processing units (GPUs)). The processor 102 may be communicatively coupled to a memory 106 via a computer bus (not depicted) responsible for transmitting electronic data, data packets, or otherwise electronic signals to and from the processor 102 and memory 106 in order to execute, implement or perform the machine-readable instructions, methods, processes, elements, or limitations, as illustrated, depicted, or described for the various flowcharts, illustrations, diagrams, figures, and/or other disclosure herein. The processor 102 may interface with the memory 106 via the computer bus to execute an operating system (OS) and/or computing instructions contained therein, and/or to access other services/aspects. For example, the processor 102 may interface with the memory 106 via the computer bus to create, read, update, delete, or otherwise access or interact with the data stored in the memory 106, database 125, and/or another source of data.

The computing environment 100 may include a network interface 104. The network interface 104 may allow the server 105 to communicate over the network 110 via any suitable wired and/or wireless connection, e.g., using any suitable network interface controller(s) of the network interface 104. The network interface 104 may include one or more transceivers (e.g., WWAN, WLAN, and/or WPAN transceivers) functioning in accordance with IEEE reference standards, 3GPP reference standards, and/or other reference standards that may be used in receipt and transmission of data via external/network ports of the server 105 connected to computer network 110.

The memory 106 may include one or more forms of volatile and/or nonvolatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others. The memory 106 may store an operating system (OS) (e.g., Microsoft Windows, Linux, UNIX, etc.) capable of facilitating the functionalities, apps, methods, or other software as described herein. The memory 106 may store one or more sets of non-transitory, computer-executable instructions that, when executed, cause the server 105 to perform certain functions.

In general, a computer program or computer based product, application, or code (e.g., the model(s), such as ML models, or other computing instructions described herein) may be stored on a computer usable storage medium, or tangible, non-transitory computer-readable medium (e.g., reference random access memory (RAM), an optical disc, a universal serial bus (USB) drive, or the like) having such computer-readable program code or computer instructions embodied therein, wherein the computer-readable program code or computer instructions may be installed on or otherwise adapted to be executed by the processor(s) 102 (e.g., working in connection with the respective operating system in memory 106) to facilitate, implement, or perform the machine readable instructions, methods, processes, elements or limitations, as illustrated, depicted, or described for the various flowcharts, illustrations, diagrams, figures, and/or other disclosure herein. In this regard, the program code may be implemented in any desired program language, and may be implemented as machine code, assembly code, byte code, interpretable source code or the like (e.g., via Golang, Python, C, C++, C#, Objective C, Java, Scala, ActionScript, JavaScript, HTML, CSS, XML, etc.).

The memory 106 may store a surgical application 108. The surgical application 108 may perform one or more functions associated with detecting zonular apparatus instability, such as analyzing a surgical image data, providing the surgical image data to one or more of models 112 (e.g., to detect zonular apparatus instability, determine a phase of the surgical procedure), execute one or more of the ML models 112 (e.g., via an ML module 122), generate outputs (e.g., user interfaces, surgical video data), generating information associated with zonular apparatus instability, etc.

The memory 106, the database 125, and/or other suitable storage may store one or more ML models 112, routines, algorithms, or other elements, referred to herein collectively at times as “models” or “machine leaning models.” Although depicted in FIG. 1 as part of memory 106, one or more of the ML models 112 may be considered as one or more computing modules 120. The ML models 112 may be referred to as receiving an input, producing or storing an output, or executing as instructions on the processor 102. Further, the ML models 112 may be stored in the memory 106 and/or the database as executable instructions, which instructions the processor 102 may retrieve from the memory 106 and/or database 125 and execute. Further, the processor 102 should be understood to retrieve from the memory 106 and/or database 125 any data necessary to perform the executed instructions (e.g., data required as an input to the routine or model), and to store in the memory 106 and/or database 125 the intermediate results and/or output of any executed instructions.

In at least one aspect, the ML models 112 may include one or more ML models, as further described below. The ML models 112 may include zonular analysis model 114 trained to determine zonular apparatus instability based upon analyzing surgical image data. The zonular analysis model 114 may include one or more of a convolutional neural network (e.g., a temporal convolutional neural network, an inflated three-dimensional convolutional neural network), a long short-term memory (LSTM), or a transformer model (e.g., a multimodal transformer). The zonular analysis model 114 may be trained using training data (e.g., stored in the training database 125A) to receive and analyze surgical image data comprising one or more images of an ophthalmic surgical procedure of the subject, and detect zonular apparatus instability within an eye of the subject depicted in the surgical image data. To detect zonular apparatus instability, the zonular analysis model 114 may identify at least one characteristic of the eye indicating zonular apparatus instability. Based upon identifying the at least one characteristic of the eye indicating the zonular apparatus instability, the zonular analysis model 114 may perform a zonular apparatus weakness analysis using the surgical image data which may indicate the severity of zonular loss, the location of zonular loss, and/or a zonular stress index (e.g., an assessment or otherwise indication of a magnitude of displacement of the lens, capsular bag, and/or zonular apparatus in response to surgical maneuvers). The zonular weakness analysis may include determining a localization or otherwise location(s) of zonular weakness (e.g., which clock hours of the zonular apparatus are involved) and/or determining the magnitude of the zonular weakness at one or more locations experiencing zonular weakness. The zonular analysis model 114 may be trained using historical surgical image data (e.g., ophthalmic surgery image data), as further described herein.

The ML models 112 may include a language model 116, such as a small language model, medium language model, large language model, hybrid language model, and/or any other suitable language model. In at least some embodiments, the language model 116 may be a generative model trained to generate information (e.g., text, audio, video, graphics, images, user interfaces) associated with the instability of the zonular apparatus. For example, the language model 116 may be designed and/or trained to understand and generate human-like language and/or text. The language model 116 may perform various natural language processing tasks and/or generate coherent and contextually relevant text. In at least some aspects, the language model 116 may operate in conjunction with, and/or be part of, a generative AI task, e.g., generating surgical support information associated with supporting an unstable zonular apparatus. The server 105 (e.g., via the surgical application 108) and/or other suitable device(s) or component(s) of the computing environment 100 may generate one or more prompts that include text, instructions, information, or other data that, when input to the language model 116, cause the language model 116 to perform one or more functions and/or tasks. The surgical application 108 may generate one or more prompts configured to cause the language model 116 to generate a surgical recommendation associated with stabling the zonular apparatus during a surgical procedure. For example, based upon the zonular apparatus weakness analysis (e.g., the localization of zonular weakness and/or determination of the magnitude of the zonular weakness), the language model 116 may generate as an output an optimal location, quantity, and type of support device (e.g., a capsular hook, a capsular tension ring, or a capsular tension segment) to use intraoperatively and/or for permanent implantation for stabilizing the zonular apparatus. It should be understood that a prompt may comprise one or more sub-prompts. Continuing with the previous example, the prompt may include one or more sub-prompts which cause the language model 116 to receive data associated with zonular apparatus weakness analysis, generate information associated with the instability of the zonular apparatus based upon the zonular apparatus weakness analysis, and output the information (e.g., as text via a display of a surgical computing device).

The ML models 112 may include a surgical phase model 118 that determines one or more phases of a surgical procedure (e.g., the ophthalmic surgical procedure) based upon analyzing the surgical image data. The surgical phase model 118 may be trained using the historical surgical image data (e.g., ophthalmic surgery image data). In an example related to a cataract ophthalmic surgery, the surgical phases may include paracentesis, medication injection, viscoelastic insertion, main wound, capsulorrhexis initiation, capsulorrhexis formation, hydrodissection, phacoemulsification, irrigation and aspiration, cortical removal, lens insertion, viscoelastic removal, and/or wound closure. In at least some embodiments, generating information associated with the instability of the zonular apparatus may be based upon a phase of the ophthalmic surgical procedure. For example, the information associated with the instability of the zonular apparatus may include a recommendation to use one or more particular surgical tools during one or more particular phases of the ophthalmic surgical procedure.

The memory 106 may store one or more computing modules 120, implemented as respective sets of computer-executable instructions (e.g., one or more source code libraries) as described herein. The computing modules 120 may include an ML module 122. In some embodiments, ML models (e.g., the ML model 112) may be applied by the ML module 122, which may include, but are not limited to linear or logistic regression algorithms, instance-based algorithms, regularization algorithms, decision trees, Bayesian networks, cluster analysis, association rule learning, artificial neural networks, deep learning, combined learning, reinforcement learning, dimensionality reduction, and support vector machines. In various embodiments, the implemented ML methods and algorithms are directed toward at least one of a plurality of categorizations of ML, such as supervised learning, unsupervised learning, and reinforcement learning. In one aspect, the ML based algorithms may be included as a library or package executed on server(s) 105. For example, libraries may include the TensorFlow based library, the Pytorch library, and/or the scikit learn Python library.

In one embodiment, the ML module 122 employs supervised learning, which involves identifying patterns in existing data to make predictions about subsequently received data. Specifically, the ML module 122 is “trained” using training data, which includes exemplary inputs and associated exemplary outputs. Based upon the training data, the ML module 122 may generate a predictive function which maps outputs to inputs and may utilize the predictive function to generate ML outputs based upon data inputs. The exemplary inputs and exemplary outputs of the training data may include any of the data inputs or ML outputs described herein. In the exemplary embodiments, a processing element may be trained by providing it with a large sample of data with known characteristics or features.

In another embodiment, the ML module 122 may employ unsupervised learning, which involves finding meaningful relationships in unorganized data. Unlike supervised learning, unsupervised learning does not involve user-initiated training based upon exemplary inputs with associated outputs. Rather, in unsupervised learning, the ML module 122 may organize unlabeled data according to a relationship determined by at least one ML method/algorithm employed by the ML module 122. Unorganized data may include any combination of data inputs and/or ML model outputs.

In yet another embodiment, the ML module 122 may employ reinforcement learning, which involves optimizing outputs based upon feedback from a reward signal. Specifically, the ML module 122 may receive a user-defined reward signal definition, receive a data input, utilize a decision making model to generate the ML output based upon the data input, receive a reward signal based upon the reward signal definition and the ML output, and alter the decision making model so as to receive a stronger reward signal for subsequently generated ML outputs. Other types of ML may also be employed, including deep or combined learning techniques.

The ML module 122 may receive labeled data at an input layer of a model having a networked layer architecture (e.g., an artificial neural network, a convolutional neural network, etc.) for training one or more ML models. The received data may be propagated through one or more connected deep layers of the ML model to establish weights of one or more nodes, or neurons, of the respective layers. Initially, the weights may be initialized to random values, and one or more suitable activation functions may be chosen for the training process. The present techniques may include training a respective output layer of the one or more ML models.

The ML module 122 may comprise a set of computer-executable instructions to implement functionality such as loading, configurating, initializing, operating, and/or storing (e.g., in the memory 106, the database 125) the ML model 112. Once trained, one or more of the trained ML model 112 may be operated in inference mode, whereupon when provided with de novo input that the model has not previously been provided, the model may output one or more predictions, classifications, etc., as described herein.

In operation, the ML module 122 may access the memory 106, the training database 125A, and/or any other data source for training data suitable to generate one or more ML models, such as the ML model 112. The training data may be sample data with assigned relevant and comprehensive labels (classes or tags) used to fit the parameters (weights) of the ML model with the goal of training it by example. In one aspect, once an appropriate ML model is trained and validated to provide accurate predictions and/or responses, the trained ML model may be loaded into the ML module 122 at runtime to process input data and generate output data.

While various embodiments, examples, and/or aspects disclosed herein may include training and generating the ML model 112 for the server 105 to load at runtime, one or more appropriately trained ML models may already exist (e.g., stored in the memory 106, the database 125) such that the server 105 may load the existing trained ML model 112 at runtime. The server 105 may retrain, fine-tune, update and/or otherwise alter an existing ML model 112 before and/or after loading the ML model 112 at runtime. Although the ML model 112 may be described as being trained and operated (e.g., via ML module 122) on the server 105, in at least one embodiment the ML model 112 may be trained on a first server 105 or other first computing device, and operated on another second server or second computing device.

In one aspect, the computing modules 120 may include an input/output (I/O) module 124, comprising a set of computer executable instructions implementing communication functions. The I/O module 124 may include a communication component configured to communicate (e.g., send and receive) data via one or more external/network port(s) to one or more networks or local terminals, such as the computer network 110 described herein. In one aspect, the server 105 may include a client-server platform technology such as ASP.NET, Java J2EE, Ruby on Rails, Node.js, a web service or online API, responsive for receiving and responding to electronic requests.

The I/O module 124 may further include or implement a user interface configured to present information to an administrator, operator or other user, and/or receive inputs from the user, such as via a touchscreen display. The I/O module 124 may facilitate I/O components (e.g., ports, capacitive or resistive touch sensitive input panels, keys, buttons, lights, LEDs), which may be directly accessible via, or attached to, the server 105 and/or may be indirectly accessible via, or attached to, another device. According to one aspect, a user may access the server 105 via a user interface to review information, make changes, input training data, initiate ML model training via the ML module 122, and/or perform other functions (e.g., operation of one or more trained models via the ML module 122).

The network 110 may include one or more networks, including a local area network (LAN), wide area network (WAN), the Internet, a combination thereof, and/or any other suitable network. Generally, the network 110 enables bidirectional communication between the server 105, the computing device 115, and other components and/or devices of the computing environment 100. In some embodiments, the network 110 may comprise a cellular base station, such as cell tower(s), communicating to the one or more components of the computing environment 100 via wired/wireless communications based upon any one or more of various mobile phone standards, including NMT, GSM, CDMA, UMTS, LTE, 5G, 6G, or the like. Additionally, or alternatively, the network 110 may comprise one or more routers, wireless switches, or other such wireless connection points communicating to the components of the computing environment 100 via wireless communications based upon any one or more of various wireless standards, including by non-limiting example, IEEE 802.11 a/ac/ax/b/c/g/n (Wi-Fi), Bluetooth, and/or the like.

The computing environment 100 may include at least one computing device 115, which may be referred to at times as a “user device.” The computing device 115 may comprise one or more computers and/or multiple, redundant, or replicated client computers accessed by one or more users. The computing device 115 may include a computer (e.g., desktop computer, laptop computer, terminal, server), a mobile device, a wearable, augmented reality glasses/headsets, virtual reality glasses/headsets, mixed or extended reality glasses/headsets, a surgical computing device (e.g., surgical computer, surgical display, surgical headset, surgical tool), and/or other suitable computing device. The computing device 115 may be associated with, for example, one or more surgical staff performing the ophthalmic surgical procedure on the subject, such as a surgical tool or camera that captures surgical image data of a surgical procedure, a surgical display that displays the surgical image data (e.g., in real-time during the surgical procedure) with additional information, a surgical headset used by a surgeon to assist with the surgical procedure (e.g., providing guidance during the surgical procedure), etc. The computing device 115 may include a processor 132 (e.g., the processor 102), a network interface 134 (e.g., the network interface 104), and a memory 136 (e.g., the memory 106. The computing device 115 may include at least one output component 140 associated with generating an output at the computing device 115. The output component 140 may include a visual component (e.g., LCD, LED, OLED, and/or head-mounted display, a light, a touchscreen, etc.), an audio component (e.g., speaker), a feedback component (e.g., haptic motor), and/or other suitable output component. The computing device 115 may include at least one input component 142 associated with receiving an input at the computing device 115, such as an a mouse, keyboard, touchscreen, a sensor, a camera (e.g., charge-coupled device, complementary metal-oxide semiconductor), and/or other suitable input component. The computing device 115 may access services, devices, and/or components of the computing environment 100 via the network 110 (e.g., transmitting data over the network 110 via the network interface 134). In some embodiments, the computing device 115 transmits to, and/or receives data from, the server 105, the database 125, and/or other components of the computing environment 100. For example, the computing device 115 may capture surgical image data via the input component 142 (e.g., camera), store the surgical image data in the patient database 125B, transmit the surgical image data to the server 105 for analysis via one or more of the models 112, etc.

In at least some embodiments, the memory 136 of the computing device 115 stores a surgical client application 138. The surgical client application 138 may be configured to provide the same and/or similar functionality as the surgical application 108. The surgical client application 138 may be in communication (e.g., via the network 110) with the surgical application 108 to provide at least some of the functionality of the surgical application 108 to the user of the computing device 115. In at least some embodiments, the surgical client application 138 may be installed on computing devices 115 used during a surgical procedure such as an ophthalmic surgical procedure, for example to provide surgical guidance, warnings, provide visual information (e.g., surgical images overlaid with information), etc.

The computing environment 100 may include at least one electronic database 125. The server 105, the computing device 115, and/or other devices and/or components of the computing environment 100 may be communicatively coupled (e.g., via network 110) to the database 125. The database 125 may include a relational database, such as Oracle, DB2, MySQL, a NoSQL database such as MongoDB, and/or another other suitable database. The database 125 may store data and/or datasets accessible to components and devices of the computing environment 100 (e.g., the server 105, the computing device 115) via the network 110. As used herein, the term “dataset” may describe sets of data composed of separate elements (e.g., files), however, the terms “data” and “dataset(s)” may be used interchangeably herein.

The database 125 may include an ML training database 125A storing training data/datasets associated with training one or more of the ML models 112. The database 125 may include a patient database 125B storing data associated with surgical patients, for example for storing surgical image data of a surgical procedure on a surgical patient in their respective patient profile in the patient database 125B.

In operation, the server 105 may obtain surgical image data comprising a plurality of images capturing an ophthalmic surgical procedure of a subject. The server 105 may obtain the surgical image data from a memory (e.g., the memory 106, the database 125), from the computing device 115 (e.g., in real-time from a surgical computing device), and/or any other suitable source of surgical image data. For example, a computing device 115 such as a surgical computer having a camera (e.g., input component 142) may record the surgical image data in substantially real-time which the computing device 115 provides to the server 105 via the network 110. The surgical application 108 executing on the server 105 may provide the surgical image data to the zonular analysis model 114 to determine the instability of the zonular apparatus based upon analyzing the surgical image data including: identifying at least one characteristic of the eye indicating zonular apparatus instability. In at least some embodiments, the characteristic of the eye indicating zonular apparatus instability may include movement of a lens, a displacement of the lens, or a depression of the lens during an associated surgical maneuver including a medication and viscoelastic instillation. In another example, the characteristic of the eye indicating zonular apparatus instability may include wrinkling of a capsular bag during a capsulotomy surgical maneuver. In yet another example, the characteristic of the eye indicating zonular apparatus instability may include one or more of deepening of an anterior chamber or backward bowing of an iris during an irrigation surgical maneuver. In still yet another example, the characteristic of the eye indicating zonular apparatus instability may include movement of a lens-capsular bag complex during a phacoemulsification surgical maneuver.

Based upon identifying the at least one characteristic of the eye indicating zonular apparatus instability, the surgical application 108 may perform a zonular apparatus weakness analysis indicating one or more of a severity of zonular loss, a location of zonular loss, or a zonular stress index. Based upon the zonular apparatus weakness analysis, the surgical application 108 may generate output data including information associated with surgical support of the zonular apparatus. The information of the output data associated with zonular apparatus instability may include a recommendation to use at least one capsular support device (e.g., a capsular hook, a capsular tension ring, or a capsular tension segment) during the ophthalmic surgical procedure, may include a surgical recommendation indicating timing associated with introducing or removing the at least one capsular support device during the ophthalmic surgical procedure, and/or may indicate a surgical risk to the subject caused by the instability of a zonular apparatus (e.g., dislocation of the capsular bag, a posterior capsule rupture, displacement of the lens, or loss of the lens behind the eye), and/or any other suitable information associated with surgical support of the zonular apparatus. The surgical application 108 may transmit the output data to the computing device 115 (e.g., via the network 110), for example causing the computing device 115 to output (e.g., via the surgical client application 138) the output data via one or more of the output components 140 (e.g., a visual output of the output data via a display, an audio output of the output data via a speaker).

In at least some embodiments, the zonular analysis model 114 and/or language model 116 may generate at least a portion of the output data. For example, the zonular analysis model 114 may generate information indicating the characteristics of the eye indicative of zonular apparatus weakness, and the language model 116 may generate surgical support recommendations (e.g., suggest the use of a particular type and/or number of capsular support devices in one or more locations of the subject's eye) based upon the zonular weakness analysis.

In at least some embodiments, the surgical phase model 118 may determine a phase of the ophthalmic surgical procedure associated with the instability of the zonular apparatus, and the output data may be based upon the determined phase of the ophthalmic surgical procedure.

Although the computing environment 100 is shown to include one each of the server 105, the network 110, and the computing device 115, it should be understood that different numbers of servers 105, networks 110 and/or computing devices 115 may be utilized. In one example, the computing environment 100 may include dozens of servers 105 all of which may be interconnected via the network 110 to hundreds of computing devices 115.

The computing environment 100 may include additional, fewer, and/or alternate components, and may be configured to perform additional, fewer, or alternate actions, including components/actions described herein. For example, although the server 105 is shown in FIG. 1 as including one instance of various components such as the processor 102, the memory 106 and the database 125, various aspects include the computing environment 100 and/or the server 105 implementing any suitable number of any of the components shown in FIG. 1 and/or omitting any suitable ones of the components shown in FIG. 1. For instance, information described as being stored in the database 125 may be stored in the memory 106, and therefore the database 125 may be omitted. Moreover, various aspects include the computing environment 100 having any suitable number of additional component(s) not shown in FIG. 1, such as but not limited to the example components described above. Furthermore, it should be appreciated that additional and/or alternative connections between components shown in FIG. 1 may be implemented. As just one example, server 105 may have a direct connection to database 125 rather than appearing to be connected via the network 110 as illustrated in FIG. 1.

### Example ML Model Training

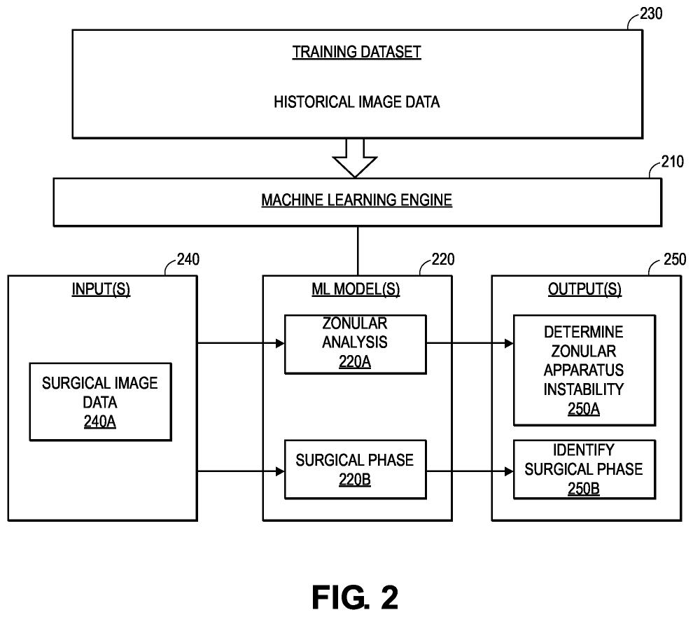

FIG. 2 depicts a combined block and logic diagram for training a machine learning model, according to embodiments. More specifically, an ML engine 210 (e.g., the ML module 122) trains one or more ML model 220 (e.g., the ML model 112) using training data 230 (e.g., stored in the training database 125A). The trained ML model 220 is applied to, and/or receives, at least one input 240 and generates at least one output 250.

The ML engine 210 may include one or more hardware and/or software components (e.g., the ML module 122) to obtain, create, operate, train, retrain, fine-tune, and/or store one or more ML models 220. It should be understood that at times the term “train” may be used interchangeably with the terms “retrain” and/or “fine-tune.” A server (e.g., the server 105), may obtain and/or have available (e.g., stored in the database 125) one or more types of training data 230 for model creation, training, retraining and/or fine-tuning (generally referred to herein as “training”). In at least one aspect, at least some of the training data 230 may be labeled to aid in training the ML model 220. The ML engine 210 may process and/or analyze the training data 230 to learn associations and/or relationships in the training data 230, and configure the ML model 220 to process the training data 230 such that when the ML model 220 receives one or more inputs 240, the ML model 220 generates appropriate output(s) 250. The ML model 220 may be trained via regression, k-nearest neighbor, support vector regression, and/or random forest algorithms, although any type of applicable ML algorithm and/or training may be used, including training using one or more of supervised learning, unsupervised learning, semi-supervised learning, and/or reinforcement learning.

In at least one aspect, the ML model 220 may be considered as successfully trained when able to achieve one or more metrics (e.g., a score indicating accuracy of the output 250) associated with its performance when processing the training data 230. Once trained, the ML engine 210 may load the ML model 220 at runtime to perform operations on one or more data inputs 240 to produce the desired data output 250.

In at least some embodiments, the ML model 220 includes a zonular analysis model 220A (e.g., the zonular analysis model 114) that may be trained via the ML engine 210 using the training data 230 to receive as an input 240 surgical image data 240A of an ophthalmic surgical procedure, and in response determine zonular apparatus instability 250A (e.g., during one or more phases of an ophthalmic surgical procedure) as the output 250.

The zonular analysis model 220A may determine zonular apparatus instability 250A based upon analyzing the surgical image data 240A to identify at least one characteristic of the eye indicating the instability of a zonular apparatus, for example during a surgical maneuver of the ophthalmic surgical procedure. In at least one embodiment, the surgical maneuver may be a medication and viscoelastic instillation and the characteristic of the eye indicative of the zonular apparatus instability may include movement of a lens, a displacement of the lens, and/or a depression of the lens. In at least one embodiment, the surgical maneuver may be a capsulotomy and the characteristic of the eye indicative of zonular apparatus instability may include wrinkling of a capsular bag. In at least one embodiment, the surgical maneuver may be an irrigation and the characteristic of the eye indicative of the instability of a zonular apparatus may include deepening of an anterior chamber and/or backward bowing of an iris. In at least one embodiment, during a phacoemulsification surgical maneuver movement of a lens-capsular bag complex may indicate zonular apparatus instability.

The training data 230 for the zonular analysis model 220A may include historical image data including images of an eye that include characteristics indicative of zonular apparatus instability (e.g., deepened anterior chamber, iris bowing, lens and/or lens-capsular bag complex movement and/or displacement, depression of the lens, capsular bag wrinkling), images of an eye that include characteristics that are not indicative of zonular apparatus instability (e.g., typical depth anterior chamber, unbowed iris, typical lens movement and/or placement, lens without depression, unwrinkled capsular bag), and/or other suitable historical images for training the zonular analysis model 220A.

In at least some embodiments, the ML model 220 includes a surgical phase model 220B (e.g., the surgical phase model 118) that may be trained via the ML engine 210 using the training data 230 to receive as an input 240 surgical image data 240A and in response surgical determine the one or more phases of the surgical procedure 250B depicted in the surgical image data 240A as the output 250. The training data 230 may include historical image data comprising a plurality of images (e.g., capturing an ophthalmic surgical procedure of a plurality of historical subjects, and/or any other suitable training data 230. At least some of the historical images may indicate or otherwise identify (e.g., via tags, labels, metadata, etc.) the phase of the surgical procedure. The surgical phase model 220B may be trained to make associations in the training data 230 between objects in the historical images that may be indicative of a phase of a surgical procedure (e.g., the condition and/or features of the eye and/or the presence or absence of particular surgical tools). For example, the surgical phase model 220B may detect an irrigation surgical tool in the images of the surgical image data 240A, and determine the phase of a cataract surgical procedure 250B is the irrigation and aspiration phase. In at least some embodiments, the surgical phase model 220B may identify the surgical phase based upon identifying other (e.g., subsequent) phases of the surgical procedure, for example subsequently identifying wound closure as the last phase of the surgical procedure based upon previously identifying the next to last phase of the surgical procedure.

The server and/or the ML engine 210 may update the training data 230 at one or more times. The ML model 220 may be retrained based upon the updated training data 230, the retrained/updated ML model 220 may be stored in memory (e.g., the memory 106) and/or subsequently executed based upon the retraining. This retraining process may cause the output 250 of the ML model 220 to improve over time. For example, the training data 230 may be updated to include new images of eyes of surgical subjects that include characteristics indicative of zonular apparatus instability such the after retraining the zonular analysis model 220A using the updated training data 230, the ability of the zonular analysis model 220A to detect or otherwise detect zonular apparatus instability 250A improves. In another example, a surgeon may provide feedback associated with zonular apparatus instability, such as feedback regarding the accuracy of the zonular apparatus weakness analysis. The updated training data 230 may incorporate the surgeon's feedback to retrain and improve the performance of the zonular analysis model 220A when performing the zonular apparatus weakness analysis.

It should be understood that functionality that is described as being associated with one or more particular models may be attributed to one or more other models. For example, the zonular analysis model 220A may be trained to identify the phase of a surgical procedure 250B based upon receiving the surgical image data 240A, such that the zonular analysis model 220A includes the functionality of the surgical phase model 220B.

### Example Generative AI

In at least some embodiments, the ML model 220 may be a generative model (e.g., the language model 116) and/or include generative functionality that allows the ML model 220 to generate new content, such as images, text (e.g., surgical guidance), or other forms of data (e.g., scripts), that is similar to, or inspired by, existing examples. Generative models operate on principles derived from machine learning, as previously described.

In some embodiments, the ML model 220 may be or include a Generative Adversarial Network (GAN). In some embodiments, GANs consist of two neural networks—the generator and the discriminator—that are trained in tandem through adversarial training. The generator aims to create data that is indistinguishable from real examples, while the discriminator's role is to differentiate between genuine and generated data. As part of the adversarial training, the generator and the discriminator iteratively compete and improve, and the generator becomes adept at producing increasingly realistic content.

In further embodiments, the ML model 220 may include and/or be trained with Recurrent Neural Networks (RNNs) or transformers, which are used for sequential data generation, such as natural language text. These models learn patterns and dependencies in the training data and can then generate new sequences by predicting the next element based on the context provided.

The present techniques may include language modeling via one or more language models (e.g., the language model 116). The language modeling may be performed one or more language models (e.g., deep learning models) trained by processing token sequences using a language model (e.g., large language model) architecture. For example, a transformer architecture may be used to process a sequence of tokens. The transformer model may include a plurality of layers including self-attention and feed-forward neural networks. The transformer architecture may enable the model to learn contextual relationships between the tokens, and to predict the next token in a sequence, based upon the preceding tokens. During training, the model is provided with the sequence of tokens and learns to predict a probability distribution over the next token in the sequence. The training process may include updating one or more model parameters (e.g., weights or biases) using an objective function that minimizes the difference between the predicted distribution and a true next token in the training data. Alternatives to the transformer architecture may include recurrent neural networks, long short-term memory networks, gated recurrent networks, convolutional neural networks, recursive neural networks, and other modeling architectures.

In at least some aspects, the ML model 220 may be a language model trained to generate surgical support information, e.g., based upon the surgical images and/or zonular apparatus instability analysis, such as surgical guidance suggesting the introductions and/or removal of capsular support devices to address zonular apparatus instability, an indication of surgical risks due to an unstable zonular apparatus, etc.

### Example System For Detecting Zonular Apparatus Instability Using Machine Learning

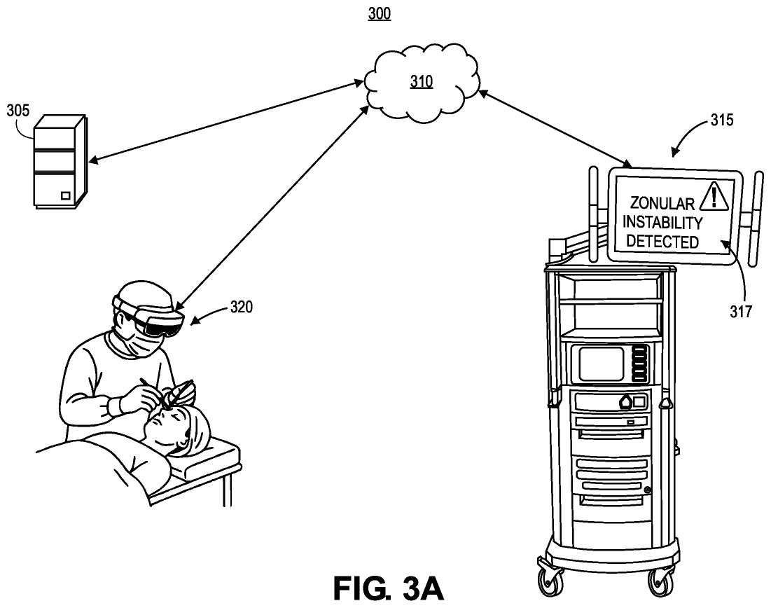

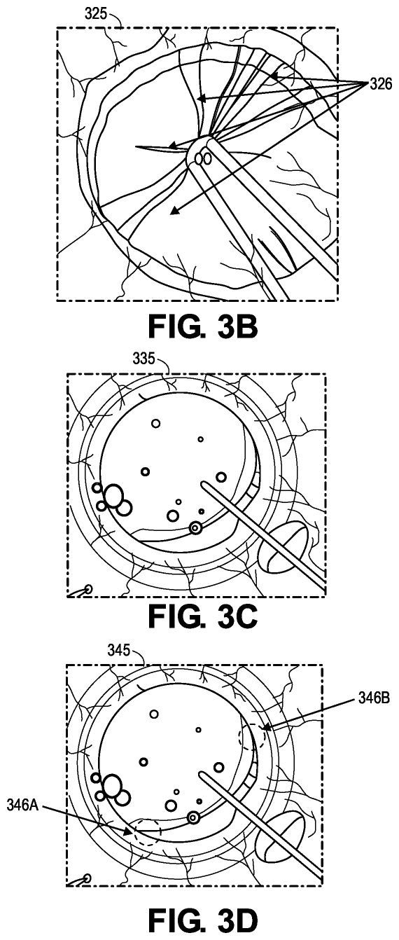

FIG. 3A depicts an example system 300 for detecting zonular apparatus instability using machine learning, according to embodiments. In general, the system 300 include devices, models, and/or components of the computing environment 100. The system 300 includes a server 305 (e.g., the server 105), a network 310 (e.g., the network 110), a surgical computer 315 (e.g., the computing device 115) having a display (e.g., the at least one output component 140, a 3D display for viewing with 3D glasses), and a surgical headset 320 (e.g., the computing device 115) worn by a surgeon performing an ophthalmic surgical procedure, such as cataract surgery, on a subject. The surgical headset 320 may include an augmented reality (AR) headset, a 3D display within the oculars of the surgical microscope, and/or any other suitable surgical display. One or more cameras (e.g., input component 142) of the surgical headset 320 and/or other cameras in the surgical environment (e.g., of a surgical tool, overhead cameras) may capture real-time surgical video (e.g., surgical image data) of the surgical procedure. The surgical headset 320 may transmit the surgical video via the network 310 to the surgical computer 315 for display, for example to provide information (e.g., surgical guidance, notifications, etc.) associated with the surgical procedure. The surgical headset 320 and/or the surgical computer 315 may analyze the surgical video via a surgical analysis application (e.g., via the surgical client application 138) to determine zonular apparatus instability and/or transmit the surgical video via the network 310 to the server 305 for analysis via a surgical analysis application (e.g., the surgical application 108).

In at least some embodiments, the server 305 may store and/or have access to one or more models (e.g., the ML models 112, 220) to analyze the surgical video to detect zonular apparatus instability. The models may include a zonular analysis model that, upon receiving the surgical video as an input from the surgical computer 315 and/or surgical headset 320, analyzes the surgical video to determine the instability of the zonular based upon identifying at least one characteristic of the subject's eye during a surgical maneuver that is indicative of zonular apparatus.

FIG. 3B depicts an example first image 325 of an ophthalmic surgical video indicating zonular apparatus instability, according to embodiments. In the first image 325, a characteristic of the eye includes wrinkles 326 on the capsular bag, for example wrinkles which appear during the capsulotomy surgical maneuver that includes puncturing the anterior capsule. The server 305 may receive the surgical video including the first image 325 from the surgical headset 320. The surgical application of the server 305 may provide the surgical video including the first image 325 to a zonular analysis model (e.g., the zonular analysis model 114, 220A) stored on, or otherwise accessible to, the server 305. The zonular analysis model may analyze the surgical video including the first image 325 and identify the wrinkles 326 as an indication of zonular apparatus weakness. Based upon detecting the wrinkles 326 in the first image 325, the zonular analysis model may perform a zonular apparatus weakness analysis based upon analyzing the surgical image data. The zonular apparatus weakness analysis may include analyzing the surgical video to evaluate spatiotemporal signs of zonular apparatus weakness that may indicate the severity and/or location of zonular loss or otherwise instability. In at least some embodiments, the zonular analysis may indicate a zonular stress index. For example, the zonular analysis model may analyze the surgical video associated with puncturing the anterior capsule during capsulotomy to evaluate characteristics of the capsular bag that indicate zonular stress, weakness and/or loss (e.g., when the wrinkles 326 appear during the capsulotomy, the quantity, contrast, and/or depth of the wrinkles 326, reaction of the capsular bag once punctured) and/or other aspects of the image (e.g., the surgical tool being used, depth of the depression of the capsular bag caused by the surgical tool) which may indicate the severity and/or location of zonular loss and/or zonular stress.

Based upon the zonular apparatus weakness analysis, the zonular analysis model may generate output data including information associated with the instability of the zonular apparatus, such as information that includes a risk to the subject based upon zonular apparatus instability (e.g., dislocation of a capsular bag, a posterior capsule rupture, a displacement of the lens, or a loss of lens material to a posterior segment), a surgical recommendation indicating (e.g., how to support the capsule intraoperatively via one or more capsular support devices such as a capsular hook, a capsular tension ring, a capsular tension segment) and/or other suitable information.

In at least some embodiments, the surgical application may provide the surgical video to a surgical phase model (e.g., the surgical phase model 118, 220B) to determine the phase of the surgical procedure. The zonular analysis model may generate the output data further based upon the phase of the ophthalmic procedure For example, based upon identifying the present phase of surgery, the output data may recommend a particular subsequent phase of the surgical procedure when the surgeon should introduce and/or remove one or more capsular support devices, such as immediately introducing a capsular hook to prevent intraocular lens dislocation and removing the capsular hook during a later phase of surgery to prevent trapping the cortex. The surgical application may cause the server 305 to transmit the output data via the network 310 to a computing device, such as the surgical computer 315 and/or the surgical headset 320, etc. For example, the surgical computer 315 may receive the output data and in response display a notification 317 on the display of the surgical computer 315 indicating zonular apparatus instability is detected and further indicate how the surgeon can mitigate any risks to the subject due to the zonular apparatus instability by employing one or more surgical tools.

FIG. 3C depicts an example second image 335 of an ophthalmic surgical video indicating zonular apparatus instability, according to embodiments. The server 305 may receive and provide surgical video including the second ophthalmic surgical image 335 to the zonular analysis model for analysis, causing the zonular analysis model 114 to determine retropulsion of the lens-iris complex in response to introduction of irrigation into the eye. In response to identifying the displaced lens, the zonular analysis model 114 may perform the zonular apparatus weakness analysis, for example determining the degree of displacement of the lens-capsular bag complex, the direction of displacement, etc. Based upon the zonular apparatus weakness analysis, the zonular analysis model 114 may generate output data that includes a recommendation to introduce two additional capsular hooks into the eye to the subject, and/or other information (e.g., an image of the eye indicating where to insert each capsular hook, information associated with the color apparatus weakness analysis such as a metric associated with a level of displacement of the lens-capsular bag complex.) The server 305 may transmit the output data via the network 310 to an output device, such as transmitting the output data to the surgical headset 320 causing the surgical headset 320 to display the recommendation to the surgeon via the AR display of the surgical headset 320, transmitting the output data to the surgical computer 315 causing the surgical computer 315 to display the output data, etc.

In at least some embodiments, the server 305, the surgical computer 315, and/or the surgical headset 320 may include a language model, such as the language model 116. The language model may be trained to generate the output data, transmit the output data (e.g., via the network 310) to another computing device, output (e.g., via visual text, spoken-word audio, an/or other suitable output) the output data via a computing device, and/or receive input (e.g., verbal prompts received via a microphone of the surgical headset 320). Accordingly, the language model may be configured to provide an interface between a user and a computing device such as the surgical computer 315 and/or surgical headset 320 executing the language model. For example, the language model may execute locally on the surgical headset 320 and be trained to provide surgical support to the user via the surgical headset 320. Accordingly, the language model may generate and/or output via the surgical headset 320 output data that includes audio which communicates a recommendation to the surgeon to introduce two additional capsular into the subject to avoid a risk of a loss of lens material to a posterior segment of the subject's eye. The surgeon may verbally request via the surgical headset 320 (e.g., via a microphone input component 142 of the surgical headset 320) that the language model generate a visual overlay of the surgical video data indicating where to insert each capsular hook in the eye of the subject. In response, the surgical client application executing on the surgical headset 320 may generate one or more prompts associated with the verbal request which cause the language model to generate one or more composite surgical images that include an augmented visual overlay on the surgical image data depicting locations for insertions of each capsular hook. The language model (e.g., via the surgical client application) may cause the surgical headset 320 to output the composite surgical images with the overlay onto the display of the surgical headset 320, transmit the composite surgical images via the network 310 for display on the surgical computer 315, etc.

FIG. 3D depicts an example composite surgical image 345 providing surgical guidance, according to embodiments. Based upon the surgeon's verbal request, the language model may generate the composite surgical image 345 (e.g., using generative artificial intelligence) that includes indicators 346A, 346B associated with suggested locations for inserting the recommended capsular hooks in the eye of the subject.

### Example Computer-Implemented Method for Determining Pupil Morphology During an Ophthalmic Surgical Procedure

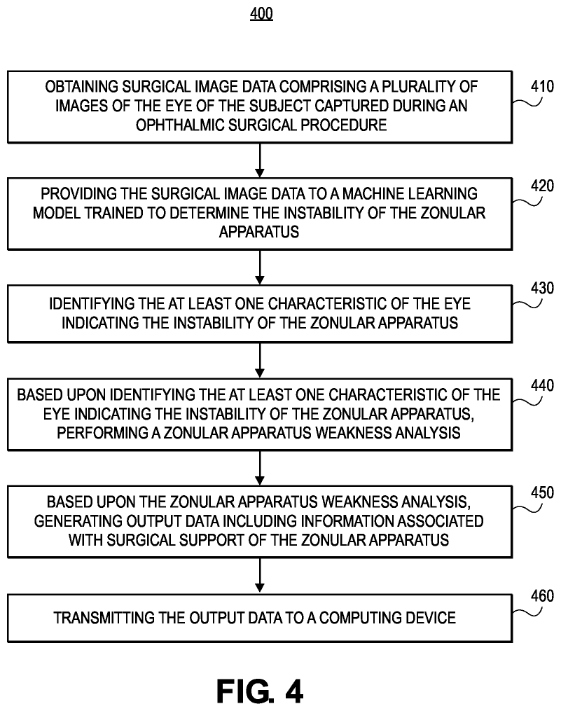

FIG. 4 is an example block flow diagram depicting a computer-implemented method 400 for detecting an instability of a zonular apparatus of an eye of a subject using machine learning, according to embodiments. In general, the computer-implemented method 400 may be performed via the computing environment 100. One or more steps of the computer-implemented method 400 may be implemented as a set of instructions stored on a computer-readable memory (e.g., the memory 106, the memory 136) and executable by one or more processors (e.g., the processor 102, the processor 132).

The computer-implemented method 400 may include obtaining surgical image data comprising a plurality of images of the eye of the subject captured during an ophthalmic surgical procedure (block 410).

The computer-implemented method 400 may include providing the surgical image data to a machine learning model (e.g., the zonular analysis model 114, 220A) trained (e.g., via the ML engine 210 and training data 230) to determine the instability of the zonular apparatus (block 420) based upon analyzing the surgical image data. Determining instability of the zonular apparatus (block 420) may include identifying at least one characteristic of the eye indicating the instability of a zonular apparatus (block 430), for example characteristic indicating zonular apparatus instability during a surgical maneuver of the ophthalmic surgical procedure. In at least some embodiments, the surgical maneuver may be a medication and viscoelastic instillation and the at least one characteristic of the eye indicative of the instability of a zonular apparatus may include one or more of movement of a lens, a displacement of the lens, or a depression of the lens. In at least some embodiments, the surgical maneuver may be a capsulotomy, and the at least one characteristic of the eye indicative of the instability of a zonular apparatus may include wrinkling of a capsular bag. In at least some embodiments, the surgical maneuver may be an irrigation and the at least one characteristic of the eye indicative of the instability of a zonular apparatus may include one or more of deepening of an anterior chamber or backward bowing of an iris. In at least some embodiments, the surgical maneuver may be phacoemulsification and the at least one characteristic of the eye indicative of the instability of a zonular apparatus may movement of a lens-capsular bag complex.

Determining instability of the zonular apparatus (block 420) may include, based upon identifying the at least one characteristic of the eye indicating the instability of the zonular apparatus, performing a zonular apparatus weakness analysis (block 440). The zonular apparatus weakness analysis may indicate one or more of a severity of zonular loss, a location of zonular loss, or a zonular stress index.

Determining instability of the zonular apparatus (block 420) may include, based upon the zonular apparatus weakness analysis, generating output data including information associated with surgical support of the zonular apparatus (block 450). In at least some embodiments, a language (e.g., the language model 116) model generates the output data. The information associated with surgical support of the zonular apparatus may include a recommendation to use at least one capsular support device (e.g., a capsular hook, a capsular tension ring, or a capsular tension segment) during the ophthalmic surgical procedure, and the at least one capsular support device may include one or more of a capsular hook, a capsular tension ring, or a capsular tension segment. The information associated with the instability of the zonular apparatus may include one or more surgical recommendations indicating timing associated with introducing the at least one capsular support device during the ophthalmic surgical procedure, or timing associated with removing the at least one capsular support device during the ophthalmic surgical procedure. The information associated with the instability of the zonular apparatus may indicate at least one surgical risk to the subject caused by the instability of a zonular apparatus, and the at least one surgical risk may include one or more of dislocation of a capsular bag, a posterior capsule rupture, a displacement of a lens, or a loss of lens material to a posterior segment.

The computer-implemented method 400 may include transmitting the output data (e.g., via the network 110, 310) to a computing device (e.g., the computing device 115, surgical computer 315, surgical headset 320) (block 460).

In at least some embodiments, the computer-implemented method 400 may include determining, via a second model (e.g., the surgical phase model 118), a phase of the ophthalmic surgical procedure associated with the instability of the zonular apparatus, wherein generating the output data (block 440) may be further based upon the phase of the ophthalmic surgical procedure.

It should be understood that not all blocks of the example flow diagram of FIG. 4 are required to be performed.

### ASPECTS

Aspect 1. A computer-implemented method for detecting an instability of a zonular apparatus of an eye of a subject using machine learning, the computer-implemented method comprising: obtaining, by one or more processors, surgical image data comprising a plurality of images of the eye of the subject captured during an ophthalmic surgical procedure; providing, by the one or more processors, the surgical image data to a machine learning model trained to determine the instability of the zonular apparatus based upon analyzing the surgical image data including: identifying at least one characteristic of the eye indicating the instability of a zonular apparatus; and based upon identifying the at least one characteristic of the eye indicating the instability of the zonular apparatus, performing a zonular apparatus weakness analysis; based upon the zonular apparatus weakness analysis, generating, by the one or more processors, output data including information associated with surgical support of the zonular apparatus; and transmitting, by the one or more processors, the output data to a computing device.

Aspect 2. The method of aspect 1, wherein the preprocessing includes extracting the region of interest corresponding to the anterior segment of the eye.

Aspect 3. The computer-implemented method of aspect 1, wherein: the at least one characteristic of the eye indicating the instability of the zonular apparatus during an associated surgical maneuver includes one or more of movement of a lens, a displacement of the lens, or a depression of the lens; and the associated surgical maneuver is a medication and viscoelastic instillation.

Aspect 4. The computer-implemented method of aspect 3, wherein the region of interest is centered on a limbus region of the eye.

Aspect 5. The method of aspect 1, wherein preprocessing further comprises resizing the surgical image data or the region of interest to preserve fine-grained anatomical structures.

Aspect 6. The method of aspect 1, wherein preprocessing further comprises normalizing pixel intensity values.

Aspect 7. The method of aspect 1, wherein preprocessing further comprises enhancing image contrast or signal-to-noise ratio.

### Additional Considerations

With the foregoing, users whose data is being collected and/or utilized may first opt-in. After a user provides affirmative consent, data may be collected from the user's device (e.g., a mobile computing device). In other embodiments, deployment and use of ML models at a client or computing device may have the benefit of removing any concerns of privacy or anonymity, by removing the need to send any personal or private data to a remote server.

The following additional considerations apply to the foregoing discussion. Throughout this specification, plural instances may implement operations or structures described as a single instance. Although individual operations of one or more methods are illustrated and described as separate operations, one or more of the individual operations may be performed concurrently, and nothing requires that the operations be performed in the order illustrated. These and other variations, modifications, additions, and improvements fall within the scope of the subject matter herein.

The patent claims at the end of this patent application are not intended to be construed under 35 U.S.C. § 114(f) unless traditional means-plus-function language is expressly recited, such as “means for” or “step for” language being explicitly recited in the claim(s). The systems and methods described herein are directed to an improvement to computer functionality, and improve the functioning of conventional computers.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment”, “in one aspect” and/or the like in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory product to which it is communicatively coupled. A further hardware module may then, at a later time, access the memory product to retrieve and process the stored output. Hardware modules may also initiate communications with input or output products, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a building environment, an office environment or as a server farm), while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a building environment, an office environment, or a server farm). In other example embodiments, the one or more processors or processor-implemented modules may be distributed across a number of geographic locations.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

Upon reading this disclosure, those of skill in the art will appreciate still additional alternative structural and functional designs for the method and systems described herein through the principles disclosed herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

Thus, many modifications and variations may be made in the techniques, methods, and structures described and illustrated herein without departing from the spirit and scope of the present claims. Accordingly, it should be understood that the methods and apparatus described herein are illustrative only and are not limiting upon the scope of the claims.

What is claimed:

1. A computer-implemented method for detecting an instability of a zonular apparatus of an eye of a subject using machine learning, the computer-implemented method comprising:
obtaining, by one or more processors, surgical image data comprising a plurality of images of the eye of the subject captured during an ophthalmic surgical procedure;
providing, by the one or more processors, the surgical image data to a machine learning model trained to determine the instability of the zonular apparatus based upon analyzing the surgical image data including:
identifying at least one characteristic of the eye indicating the instability of a zonular apparatus; and
based upon identifying the at least one characteristic of the eye indicating the instability of the zonular apparatus, performing a zonular apparatus weakness analysis;
based upon the zonular apparatus weakness analysis, generating, by the one or more processors, output data including information associated with surgical support of the zonular apparatus; and
transmitting, by the one or more processors, the output data to a computing device.

2. The computer-implemented method of claim 1, wherein:
the at least one characteristic of the eye indicating the instability of the zonular apparatus during an associated surgical maneuver includes one or more of movement of a lens, a displacement of the lens, or a depression of the lens; and
the associated surgical maneuver is a medication and viscoelastic instillation.

3. The computer-implemented method of claim 1, wherein:
the at least one characteristic of the eye indicating the instability of the zonular apparatus during an associated surgical maneuver includes wrinkling of a capsular bag; and
the associated surgical maneuver is a capsulotomy.

4. The computer-implemented method of claim 1, wherein:
the at least one characteristic of the eye indicating the instability of the zonular apparatus during an associated surgical maneuver includes one or more of deepening of an anterior chamber or backward bowing of an iris; and
the associated surgical maneuver is an irrigation.

5. The computer-implemented method of claim 1, wherein:
the at least one characteristic of the eye indicating the instability of the zonular apparatus during an associated surgical maneuver includes movement of a lens-capsular bag complex; and
the associated surgical maneuver is a phacoemulsification.

6. The computer-implemented method of claim 1, wherein the zonular apparatus weakness analysis indicates one or more of a severity of zonular loss, a location of zonular loss, or a zonular stress index.

7. The computer-implemented method of claim 1, further comprising:
determining, by the one or more processors via a second model, a phase of the ophthalmic surgical procedure associated with the instability of the zonular apparatus,
wherein generating the output data is further based upon the phase of the ophthalmic surgical procedure.

8. The computer-implemented method of claim 1, wherein:
the information associated with the instability of the zonular apparatus includes a recommendation to use at least one capsular support device during the ophthalmic surgical procedure; and
the at least one capsular support device includes one or more of a capsular hook, a capsular tension ring, or a capsular tension segment.

9. The computer-implemented method of claim 8, wherein the information associated with the instability of the zonular apparatus includes one or more surgical recommendations indicating timing associated with introducing the at least one capsular support device during the ophthalmic surgical procedure, or timing associated with removing the at least one capsular support device during the ophthalmic surgical procedure.

10. The computer-implemented method of claim 1, wherein:
the information associated with surgical support of the zonular apparatus indicates at least one surgical risk to the subject caused by the instability of a zonular apparatus; and
the at least one surgical risk includes one or more of dislocation of a capsular bag, a posterior capsule rupture, a displacement of a lens, or a loss of lens material to a posterior segment.

11. The computer-implemented method of claim 1, wherein a language model generates the output data.

12. A system for detecting an instability of a zonular apparatus of an eye of a subject using machine learning, the system comprising:
one or more processors; and
one or more non-transitory memories storing processor-executable instructions that, when executed by the one or more processors, cause the system to:
obtain surgical image data comprising a plurality of images of the eye of the subject captured during an ophthalmic surgical procedure;
provide the surgical image data to a machine learning model trained to determine the instability of the zonular apparatus based upon analyzing the surgical image data including:
identifying at least one characteristic of the eye indicating the instability of a zonular apparatus; and
based upon identifying the at least one characteristic of the eye indicating the instability of the zonular apparatus, performing a zonular apparatus weakness analysis;
based upon the zonular apparatus weakness analysis, generate output data including information associated with surgical support of the zonular apparatus; and
transmit the output data to a computing device.

13. The system of claim 12, wherein:
the at least one characteristic of the eye indicating the instability of the zonular apparatus during an associated surgical maneuver includes one or more of movement of a lens, a displacement of the lens, or a depression of the lens; and
the associated surgical maneuver is a medication and viscoelastic instillation.

14. The system of claim 12, wherein:
the at least one characteristic of the eye indicating the instability of the zonular apparatus during an associated surgical maneuver includes wrinkling of a capsular bag; and
the associated surgical maneuver is a capsulotomy.

15. The system of claim 12, wherein:
the at least one characteristic of the eye indicating the instability of the zonular apparatus during an associated surgical maneuver includes one or more of deepening of an anterior chamber or backward bowing of an iris; and
the associated surgical maneuver is an irrigation.

16. The system of claim 12, wherein:
the at least one characteristic of the eye indicating the instability of the zonular apparatus during an associated surgical maneuver includes movement of a lens-capsular bag complex; and
the associated surgical maneuver is a phacoemulsification.

17. The system of claim 12, further comprising instructions that, when executed by the one or more processors, cause the system to:
determine, via a second model, a phase of the ophthalmic surgical procedure associated with the instability of the zonular apparatus,
wherein generating the output data is further based upon the phase of the ophthalmic surgical procedure.

18. The system of claim 12, wherein:
the information associated with the instability of the zonular apparatus includes a recommendation to use at least one capsular support device during the ophthalmic surgical procedure; and
the at least one capsular support device includes one or more of a capsular hook, a capsular tension ring, or a capsular tension segment.

19. The system of claim 18, wherein the information associated with the instability of the zonular apparatus includes one or more surgical recommendations indicating timing associated with introducing the at least one capsular support device during the ophthalmic surgical procedure, or timing associated with removing the at least one capsular support device during the ophthalmic surgical procedure.

20. The system of claim 12, wherein:
the information associated with surgical support of the zonular apparatus indicates at least one surgical risk to the subject caused by the instability of a zonular apparatus; and
the at least one surgical risk includes one or more of dislocation of a capsular bag, a posterior capsule rupture, a displacement of a lens, or a loss of lens material to a posterior segment.

21. A non-transitory computer readable storage medium storing computer-executable instructions that, when executed by one or more processors, cause the one or more processors to at least:
obtain surgical image data comprising a plurality of images of an eye of a subject captured during an ophthalmic surgical procedure;
provide the surgical image data to a machine learning model trained to determine instability of a zonular apparatus of the eye based upon analyzing the surgical image data including:
identifying at least one characteristic of the eye indicating the instability of a zonular apparatus; and
based upon identifying the at least one characteristic of the eye indicating the instability of the zonular apparatus, performing a zonular apparatus weakness analysis;
based upon the zonular apparatus weakness analysis, generate output data including information associated with surgical support of the zonular apparatus; and
transmit the output data to a computing device.

22. The method of claim 1, further comprising obtaining, by the one or more processors, surgical image data including a plurality of images of the eye of the subject captured during an ophthalmic surgical procedure, wherein the surgical image data comprises a sequence of temporally ordered images.

23. The method of claim 1, further comprising performing, by the one or more processors, preprocessing on the surgical image data, including extracting a region of interest corresponding to an anterior segment of the eye and at least one of resizing the surgical image data or the region of interest, normalizing pixel intensity values, or enhancing image contrast or quality.

24. The method of claim 1, further comprising providing, by the one or more processors, the surgical image data to a machine learning model trained to determine the instability of the zonular apparatus based upon analyzing the surgical image data, including analyzing temporal information across the sequence of images to determine motion, persistence, or displacement of one or more anatomical structures.
